(19) United States
(12) Patent Application Publication
(10) Pub. No.: US 2014/0369997 A1
(43) Pub. Date: Dec. 18, 2014

(54) Pharmaceutical composition for a foaming eye drop

(71) Applicant: Calvin Alexander Grant, Oak Park, IL (US)

(72) Inventor: Calvin Alexander Grant, Oak Park, IL (US)

(73) Assignee: PASE, OAK PARK, IL (US)

(21) Appl. No.: 13/385,123

(22) Filed: Feb. 2, 2012

Related U.S. Application Data
(60) Provisional application No. 61/463,378, filed on Feb. 16, 2011.

(51) Int. Cl. A61K 47/26; A61K 31/713; A61K 39/395; A61K 38/02; A61K 9/12; A61K 45/06

(52) U.S. Cl. CPC A61K 47/26; A61K 9/122; A61K 45/06; A61K 39/39533; A61K 38/02; A61K 31/713
USPC 424/130.1; 536/95; 514/781; 514/20.8; 514/44 R; 530/387.1; 530/350; 536/23.1

(57) ABSTRACT

The present application describes a method of creating a foaming ophthalmic formulation. The described formulation includes the addition of a foaming agent. The resulting solution is designed to be distributed by a non-aerosol foaming bottle/or aerosol.

### CROSS-REFERENCE TO RELATED APPLICATIONS

The present application claims priority benefit under 35 U.S.C. §119(e) of U.S. Provisional Patent Application Ser. No. ______ filed ______. The disclosure of this application is incorporated herein by reference.

### FIELD OF INVENTION

The present invention is in the field of pharmaceuticals, more specifically a pharmaceutical composition for a foaming eye drop.

### SUMMARY OF INVENTION

The present invention is a novel pharmaceutical composition for a foaming eye drop. The medication is formulated with one or more foaming agents. These agents allow the solution to form foam when mixed with a gas and typically increase the viscosity of the solution. Most medication for the eye today is in the aqueous form and delivered by an eye dropper. Despite most people's familiarity with the eye dropper, many find it difficult to use and often apply too little medication by misplacing the drop or too much medication by distributing more than one drop at time. Recent studies have found that 61% to 90% of patients with glaucoma, one of the largest eye drop user groups, are unable to correctly use the standard dropper.

The dropper delivers 50-70 microliters of medication in a single drop. Even if the drop lands in the in correct position, the natural tendency of the eye is to immediately reduce the volume to 7 microliters. The result of this is that 86% to 90% of all eye medication today is wasted. Innovations that have been made to improve this design have often just attempted to improve the delivery of the drop to the eye; products such as Xal-Ease™, VersiDoser™ and Visine® Pure Tears Single Drop dispenser are examples of this. These methods still do not solve the problem that the liquid drop is an inefficient method of delivery of medication to the eye.

The present invention changes the pharmaceutical formulation of topical eye medication so that it is capable of being dispensed as a foam rather than a liquid. By foaming the eye medication several benefits can be obtained, one of the most important of which is the reduction of wasted medication. This is achieved because the foam can reach a sufficient volume to form a drop and fall into the eye without exceeding the amount of fluid that they eye can actually hold. In other words, using the present invention the actual volume of liquid medication that is administered per drop can match what the eye can actually hold since the majority of the volume of the drop is comprised of gas. The reduction in volume dispensed to the eye will also reduce the rate at which the eye replaces the tear film. This will allow the medication to remain in contact with the eye for a longer amount of time and thus increase the bioavailability of the drug.

By foaming the drop, the drop also becomes larger and easier to apply to the eye. Another significant advantage of the present invention is that the gas that is used to create the foam can be used to augment the effectiveness of the drop. The foaming drop also has the added benefits of comfort of use, ease of application, and higher patient compliance. The invention described here is a more effective and more efficient method of delivery of medication to the eye.

### DESCRIPTION OF INVENTION

The present invention is a pharmaceutical composition for eye medication that is to be delivered as a foam. This is not a formula for a specific medication, but rather a method to change the pharmaceutical composition of any type of medication that is meant to be topically applied to the eye, so that it may be applied as a foam. The pharmaceutical composition of the eye drop must be modified to allow the fluid when mixed with some type of gas to form a stable foam. This is best achieved by addition of one or more foaming agents. There are a variety of foaming agents from which to choose. It is best to choose the agent that is able to augment the effectiveness of the active ingredient. Considerations should also be given to the stability of the solution. Foaming agents include but are not limited to:
locust bean gum, sodium alginate, sodium caseinate, albumin, bovine serum albumin, egg albumin, human serum albumin, synthetic albumin, carbohydrates, corneal penetration enhancers, carrageenan gum, cellulose derivatives, cyclodextrin, ethylene glycol, propylene glycol , polyethylene glycol , detergents, gelatin, agar, gycerine, carrageenin gum, sodium alginate, xanthan gum, quince seed extract, tragacanth gum, gellan gum, guar gum, gelatin, cationic guars, hydroxypropyl guar gum, starch, an amine-bearing polymer, chitosan, alginic acid, hyaluronic acids and salts, chemically modified starches, lipids, carboxyvinyl polymers, polyvinylpyrrolidone, polyvinyl alcohol, a polyacrylic acid polymer, a polymethacrylic acid polymer, polyvinyl acetate, polyvinyl chloride polymers, polyvinylidene chloride polymers, polyols, methylcellulose, hydroxypropyl cellulose, hydroxypropyl methylcellulose, hydroxyethyl cellulose, hydroxypropylmethyl cellulose, hydroxypropymethylcellulose, hydroxyethycellulose, methylhydroxyethylcellulose, methylhydroxypropylcellulose, hydroxyethylcarboxymethylcellulose, carboxymethyl cellulose, carboxymethylhydroxyethylcellulose, cationic cellulose, PEG 1000, PEG 4000, PEG 6000, PEG 8000, surfactants, non-ionic surfactants, sucrose, and trehalose.

The preferred embodiment is a solution containing between 0.25%-5% of hydroxypropyl methylcellulose. This has been proven to produce a foam that is capable of holding enough gas to result in a 90% reduction in volume of the medication distributed in a drop. In other words the foam that is created is 90% gas and 10% liquid. The addition of hydroxypropyl methylcellulose is one of many ways that the pharmaceutical composition of the eye medication can be changed.

Any preferred additive should have properties similar to hydroxypropyl methylcellulose such that a large reduction in volume of liquid from the foamed form to the liquid form is obtained. The additive should be chosen to reduce the risk of eye irritation and to minimize any potential binding of the active ingredient. Hydroxypropyl methylcellulose is especially preferred because it is already known to be a safe ingredient to be applied to the eye as it is currently used in many eye drops to lubricate the eye. It also produces a more viscous solution which increases the retention time in the eye, thus giving the medication more time to be absorbed. The use of albumin has also been tested and found to be an excellent foaming agent. An albumin concentration between 1%-25% has been found to produce foam of the desired consistency.

The use of a non-ionic surfactant that is a known corneal penetration enhancer such as laurocapram, is also an excellent choice especially when the active ingredient is hydrophilic and would benefit from increased corneal absorption. The use of a penetration enhancer as the foaming component of the composition has the benefit of further lowering the required active ingredient as substances such as laurocapram has been shown to greatly increase corneal penetration for hydrophilic molecules. Examples of other enhancers include but are not limited to hexamethylene lauramide, hexamethylene octanamide, and decylmethy sulfoxide. Careful consideration should be taken when formulating the pharmaceutical composition in order to ensure that the foaming ingredient properly enhances the active ingredient to gain the greatest benefit. Mucoadhesive polymers and in situ gelling excipients have also been found to further increase drug bioavailability.

The present pharmaceutical composition is an effective method to deliver both fluid and gas to the eye. The active ingredients can either be contained in the fluid, the gas, or both. The gas that comprises this foam provides opportunity for further therapeutic use that a normal eye drop does not. Air from the surrounding area will be suitable for most applications. In distributions where the sterility is of utmost importance, filtered air may be used. Other possible applications include the use of inert gasses such as carbon dioxide or medicinal gases. In most applications of the present invention the active ingredient will be either dissolved or suspended in the fluid. However the gas that is used to form the foam can also be therapeutic. This type of distribution would use medicinal gases such as nitrous oxide. When a gas is used to provide a medical benefit, it can be used in combination with an aqueous active ingredient or it can be used just with the foaming agent.

The pharmaceutical composition described above will need to be delivered through a device that is capable of creating a foam. This can be achieved in various ways. The preferred method is by a foam dispensing pump container. Suitable pump containers include U.S. Pat. Nos. 5,271,530, 5,429,279, and 5,337,929. The foaming pump described in U.S. patent application Ser. No. 0096412 is especially suitable due to its ability to function while inverted. These containers could easily be modified to better suit ophthalmic delivery. Desired modifications include changing the nozzle head of the pump to resemble a typical eye dropper and reducing the size of container. These foaming mechanisms use air from the surrounding area but they could also be modified to use any type of gas.

The present invention is capable of being used in an aerosol device as well. When the pharmaceutical composition is foamed using an aerosol device, any type of gas such as medicinal gases or inert gases may be used. When an aerosol is used the medication and or foaming agent is under pressure in liquid form along with the propellant. The use of an aerosol as the delivery method also has the advantage of reducing the need for a preservative. The composition can be formulated just as described above. The can of the aerosol container would preferably be of a smaller size to suit the dosage that is common in most eye drops and the nozzle which supplies the foam should be modified to make application to the eye easier. Another possible delivery method for this device is a pressurized gas canister with hose leading to an applicator that mixes the gas and fluid to create foam. This type of delivery system would be especially useful in the surgical environment. The methods of delivery described here is certainly not a complete list. The presented pharmaceutical composition can be used with any type of device that is capable of mixing a fluid and gas in order to produce a foam.

The pharmaceutical composition described here works with a very wide range of medicines for the eye. Table 1 lists specific examples of the medications and table 2 lists active ingredients that would be suitable for this type of delivery. Tables 1 and 2 are by no means to be interpreted as complete lists, as this composition could be used in any type of medication that is applied directly to the eye. Many future medications that are not currently on the market could also benefit from the present invention.

While this composition has many advantages that all eye medications would benefit from such as easier administration and increased contact time with the eye, the drugs that will see the most benefit are drugs that have costly active ingredients. These types of costly medications will greatly benefit from the fact that much less active ingredient is necessary to produce the same result. The reduction in active ingredient is a result of a lower amount of dispensed per application and increased corneal absorption from increased contact time and/or penetration enhancers. Some of the foaming agents listed above are currently excipients in extended release formulations, which reduce the number of applications required per day. This would further reduce the amount of medication needed.

While the previous written description of the invention and embodiments enables one of ordinary skill to make and use the present invention, those will understand and appreciate the existence of variations, combinations and equivalents of the specific embodiment, method, and examples herein. The invention should therefore not be limited by the above described embodiment, method, and examples, but by all embodiments and methods within the scope and spirit of the invention as claimed.

What is claimed is:

1. A foaming ophthalmic pharmaceutical composition including a foaming agent to be topically applied to the eye from a container capable of distributing foam.

2. A pharmaceutical composition of claim 1 wherein the composition further comprises of a pharmaceutically active ingredient.

3. A pharmaceutical composition of claim 1 wherein the foaming agent is one or more of the following in a concentration capable of creating a foam: locust bean gum, sodium alginate, sodium caseinate, albumin, bovine serum albumin, egg albumin, human serum albumin, synthetic albumin, carbohydrates, corneal penetration enhancers, carrageenan gum, cellulose derivatives, cyclodextrin, ethylene glycol, propylene glycol , polyethylene glycol , detergents, gelatin, agar, gycerine, carrageenin gum, sodium alginate, xanthan gum, quince seed extract, tragacanth gum, gellan gum, guar gum, gelatin, cationic guars, hydroxypropyl guar gum, starch, an amine-bearing polymer, chitosan, alginic acid, hyaluronic acids and salts, chemically modified starches, lipids, carboxyvinyl polymers, polyvinylpyrrolidone, polyvinyl alcohol, a polyacrylic acid polymer, a polymethacrylic acid polymer, polyvinyl acetate, polyvinyl chloride polymers, polyvinylidene chloride polymers, polyols, methylcellulose, hydroxypropyl cellulose, hydroxypropyl methylcellulose, hydroxyethyl cellulose, hydroxypropylmethyl cellulose, hydroxypropymethylcellulose, hydroxyethycellulose, methylhydroxyethylcellulose, methylhydroxypropylcellulose, hydroxyethylcarboxymethylcellulose, carboxymethyl cellulose, carboxymethylhydroxyethylcellulose, cationic cellulose, PEG 1000, PEG 4000, PEG 6000, PEG 8000, surfactants, non-ionic surfactants, sucrose, and trehalose.

4. A pharmaceutical composition of claim 1 wherein the composition further comprises of one or more pharmaceutically active ingredients.

5. A pharmaceutical composition of claim 1 wherein the composition further comprises of one or more pharmaceutically active ingredients from the following:
acyclovir,antazoline,apraclonidine,atropine,azelastine,bacitracin,belladonna,bepotastine besilate, betaxolol, bimatoprost, bleph-10, brimonidine tartrate, brinzolamide, bromfenac, carboxyl methylcellulose, carboxymethylcellulose, carboxymethylcellulose, carteolol, ciprofloxacin hydrochloride, coniummaculatum, cromolyn, cromolynsodium, cyclopentolate, cyclosporineopthalmic emulsion, dexamethasone, dextran, diclofenac, dipivefrin, dorzolamide, dorzolamide HCL, emedastine, erythromycin, euphrasia, famciclovir, fluorometholone, flurbiprofen, gatifloxacin, gentamicin, gentamycin, glycerin, glycerin, gramicidin, hepar sulphuris, homatropine, hydroxypropylmethylcellulose, isoptoatropine, isoptohomatropine, ketorolac, ketotifenfumarate, latanoprost, levobunolol, levocabastine, light mineral oil, lodoxamidetromethamine, loteprednol, loteprednoletabonate, mineral oil, moxifloxacinHCL, multivitamin, N-Acetyl-L-Carnosine, naphazoline, naphazolineHCL, naphazolineHydrochloride, natriummuriaticum, nedocromil,neomycin, neomycinsulfate, nepafenac, norfloxacin, ofloxacin, olopatadine, oxymetazolineHCL, parrafin, pectin, pemirolast, petrolatum, pheniramine, pheniraminemaleate, phenylephrine, phenylephrineHCL, pilocarpine, polyethyleneglycol, polyethylene glycol 400, polymixin, polymixin B,polymyxin B sulfate, polysorbate 80, polyvinyl alcohol (PVA), povidone, prednisolone, prednisone, proparacaine, proparacaine hydrochloride, propyleneglycol, rimexolone, rutagraveolens, sodium chloride, sodium sufacetamide, soylecithin, sulamyd, sulfacetamide, tetracaine, tetrahydrozoline HCL, tetrahydrozolineHydrochloride, tetrahydrozyline, timolol, timolol maleate, tobramycin, trifluridine, trimethoprim, tropicamide, unoprostone, valacyclovir, white perolatum, white petroleum, and zinc sulfate.

6. A pharmaceutical composition of claim 1 wherein the foaming agent is hydroxypropyl methylcellulose (hypromellose) in a concentration between 0.25%-5%.

7. A pharmaceutical composition of claim 1 wherein the foaming agent is naturally derived or created albumin in a concentration between 1%-25%.

8. A pharmaceutical composition of claim 1 wherein the container is foaming pump

9. A pharmaceutical composition of claim 1 wherein the container is an aerosol.

10. An ophthalmic pharmaceutical composition including a foaming agent intended to be topically applied to the eye from an aerosol container mixing the composition with gas to form foam.

11. A pharmaceutical composition of claim 10 wherein the gas is or contains a pharmaceutically active ingredient.

12. Any recombinant DNA, antibody, protein for medicinal purposes in 3 is covered.
